(19) United States
(12) Patent Application Publication
(10) Pub. No.: US 2019/0083711 A1
(43) Pub. Date: Mar. 21, 2019

(54) Medical and Dental Safety Syringe

(71) Applicants: Khurrum Masood Ashraf, Waterloo (CA); Bruce Zagrodney, Scarborough (CA)

(72) Inventors: Khurrum Masood Ashraf, Waterloo (CA); Bruce Zagrodney, Scarborough (CA)

(21) Appl. No.: 16/136,901

(22) Filed: Sep. 20, 2018

Related U.S. Application Data
(60) Provisional application No. 62/560,946, filed on Sep. 20, 2017.

(51) Int. Cl. A61M 5/315; A61M 5/31; A61M 5/24; A61M 5/20; A61M 5/32

(52) U.S. Cl. CPC A61M 5/31513; A61M 5/3148; A61M 5/3202; A61M 5/31571; A61M 5/2033; A61M 5/2425

(57) ABSTRACT

A plunging unit for a syringe is provided comprising (i) a plunger comprising a plunger head connected to a thumb rest by a shaft; and (ii) a pusher housing comprising a finger rest and an internal channel connecting a plunger-receiving end of the housing to a barrel-receiving end of the housing, wherein the plunger-receiving end is adapted to receive the plunger head into the channel and the barrel-receiving end comprises one or more protrusions adapted to engage a barrel of a carpule housing unit. The plunging unit optionally includes an aspirator adapted to cause a backward movement of the plunger away from a site of injection. The aspirator may comprise an actuator ring on the shaft of the plunger adjacent to the plunger-receiving end of the housing, and when the actuator ring is moved in a backward direction, a spring-loaded piston that sits within the pusher housing and houses the shaft of the plunger is pulled backward which causes the plunger to move backward.

### FIELD OF THE INVENTION

The present invention relates to a medical device and more particularly to a medical and dental safety syringe.

### BACKGROUND ART

Syringes are used in the fields of medicine and dentistry to dispense medicine to patients. One specialized class of syringes is especially adapted for use in dentistry, particularly for desensitizing or anaesthetizing the patient's intraoral tissues with an anesthetic prior to having dental work performed.

This class of syringes is constructed so that they can be loaded with cartridges pre-filled with medication, also called carpules. A typical carpule has: (i) a barrel containing the medication; (ii) a seal or stopper at the rear end of the barrel; and (iii) a penetrable membrane at the front end of the barrel. Carpules are loaded into a hollow housing of the syringe.

Dental syringes generally include a plunger unit attached to the rear end of the hollow housing of the syringe. The plunger unit includes: (i) a plunger for engaging the seal of the carpule; (ii) a circular thumb ring for driving and retracting the plunger; and (iii) a shaft connecting the plunger to the circular thumb ring. The circular thumb ring provides some additional control of the plunger over the conventional T-shaped finger flange present on most medical syringes, particularly for retracting the plunger. The plunger unit may also include a finger rest for the index finger and middle finger situated between the circular thumb ring and the plunger and a round lever or button situated above the finger rest which activates the syringe to aspirate fluid from the injection site. One would use his or her thumb to push this lever. This is important in the field of dentistry because after an initial stick, the dentist needs to determine if a blood vessel has been hit. It is not desirable to inject an anesthetic into a blood vessel because epinephrine in the anesthetic can have a detrimental effect on the heart rate. Furthermore, the anesthetic would be carried away from the injection site and into the bloodstream, and the operating site would not be sufficiently anesthetized. By pushing the lever or button, the dentist can determine if a blood vessel has been hit by whether or not blood has been pulled into the carpule by aspiration. If a blood vessel has been hit, the dentist will need to find another injection site.

A double-ended hypodermic needle is attached to the front end of the hollow housing of the syringe. The rear end of the needle penetrates the membrane on the front end of the carpule. The front end of the needle is used to stick into the injection site of the patient. When not in use, a plastic cover is placed over the front end of the needle. Prior to use, the plastic cover is manually removed, thus exposing the front end of the needle.

When in use, the dentist uses the circular thumb ring to depress the plunger. The plunger engages and moves the carpule seal into the interior of the barrel, which drives the medication out the front end of the carpule and into the needle. The medication is delivered to the patient through the front end of the needle.

The circular thumb ring provides better control of depression and retraction of the plunger than the conventional T-shaped finger flange. However, dentists and dental assistants still find that the circular thumb ring does not fit their thumb tips very well and during the course of a work day, develop fatigue because of this awkward fit. Many times, the user's thumb can get caught in the thumb ring and due to loss of control, the patient or user is at risk of accident. Improvements are desired to increase control of the plunger and reduce fatigue.

Furthermore, the manual removal and replacement of the plastic cover over the needle leads to many accidental needle sticks which injure dentists, dental assistants, patients and others. This danger is magnified by the risk of the needle being contaminated with bacteria and/or viruses, thus leading to the spread of communicable diseases.

Thus, it is desired to provide an improved system to address at least one of the disadvantages of current medical and dental syringes.

### SUMMARY OF THE INVENTION

A novel plunging unit for use in a syringe has been developed.

In one aspect, the present invention provides a plunging unit for a syringe comprising:

(i) a plunger comprising a plunger head connected to a thumb rest by a shaft;

(ii) a pusher housing comprising a finger rest and an internal channel connecting a plunger-receiving end of the housing to a barrel-receiving end of the housing, wherein the plunger-receiving end is adapted to receive the plunger head into the channel and the barrel-receiving end comprises one or more protrusions adapted to engage a barrel of a carpule housing unit; and

(iii) optionally, an aspirator adapted to cause a backward movement of the plunger away from a site of injection, wherein the aspirator comprises an actuator ring on the shaft of the plunger adjacent to the plunger-receiving end of the housing, and when the actuator ring is moved in a backward direction, a spring-loaded piston that sits within the pusher housing and houses the shaft of the plunger is pulled backward which causes the plunger to move backward.

In another aspect, a kit is provided comprising a plunging unit as defined above, and one or more housing units, wherein the housing units comprise:

i) a barrel adapted to receive a carpule, said barrel having a front end comprising a needle mounting means, and a rear end comprising an opening to receive a carpule and attachment means to releasably engage with the protrusions on pusher housing of the plunging unit; and

ii) a sheath having a front end and a rear end, said sheath being slidably mounted onto the barrel, said sheath being moveable between a first extended position to cover a needle mounted on the barrel and a second retracted position which exposes a needle mounted on the barrel.

These and other aspects of the invention are described by reference to the detailed description and the following figures.

### BRIEF DESCRIPTION OF THE DRAWINGS

FIG. 1 illustrates a perspective view of syringe in accordance with an embodiment of the present invention.

FIG. 2 illustrates a cut-away view of the syringe of FIG. 1.

FIG. 3 illustrates a cut-away view of a housing unit for a syringe in an embodiment.

FIG. 4 illustrates a cut-away view of a barrel of the housing unit of FIG. 3 attached to a plunger unit in an embodiment.

FIG. 5 illustrates a plunger unit for a syringe in an embodiment,

FIG. 6 illustrates a perspective view of a plunger unit in an embodiment.

FIG. 7 illustrates an exploded view of a syringe including a plunger unit and a housing unit in accordance with an embodiment of the invention.

### DETAILED DESCRIPTION OF THE INVENTION

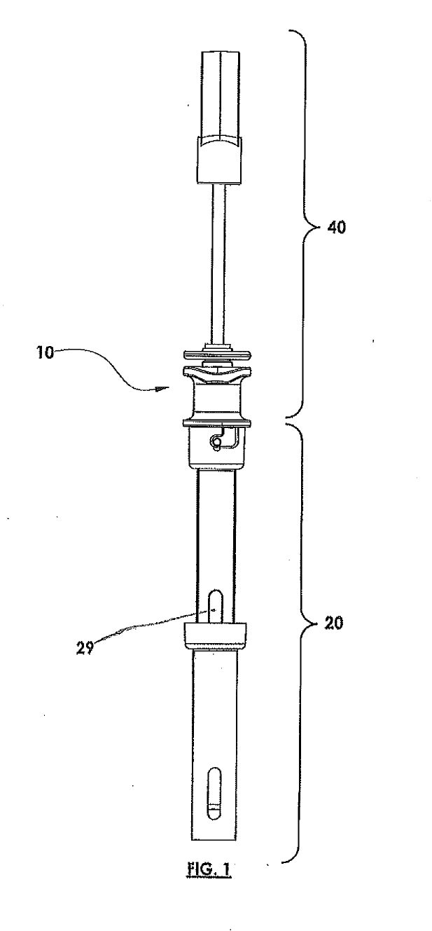

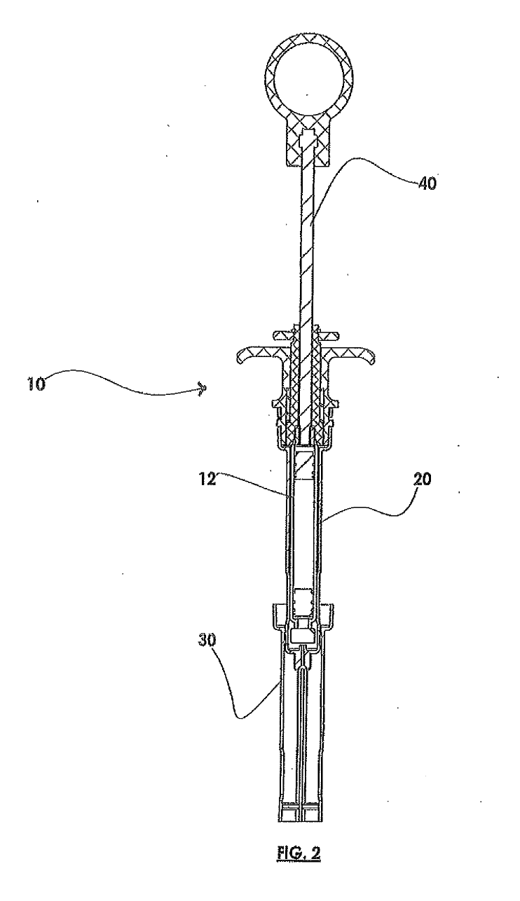

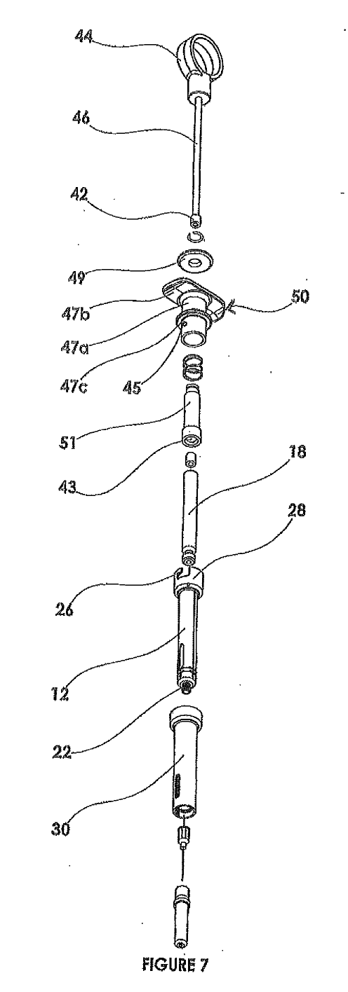

An improved medical/dental syringe is provided. A preferred embodiment of the medical and dental syringe 10 of the present invention is illustrated in FIG. 7. The syringe comprises a tubular carpule housing unit 20 and a plunger unit 40 for use with a needle unit as shown in FIGS. 1 and 2.

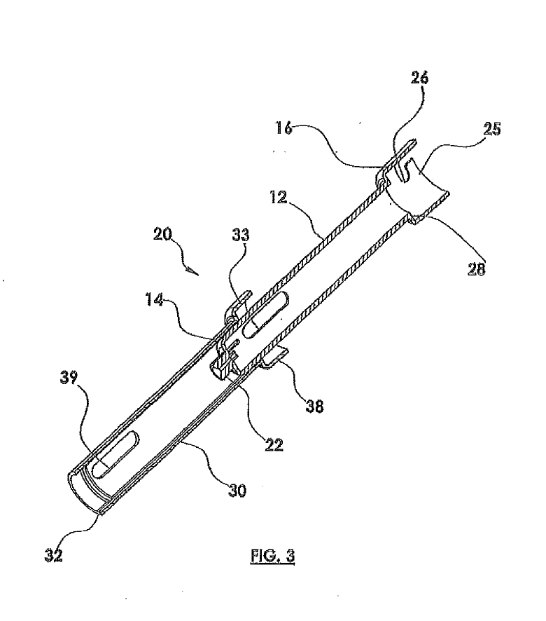

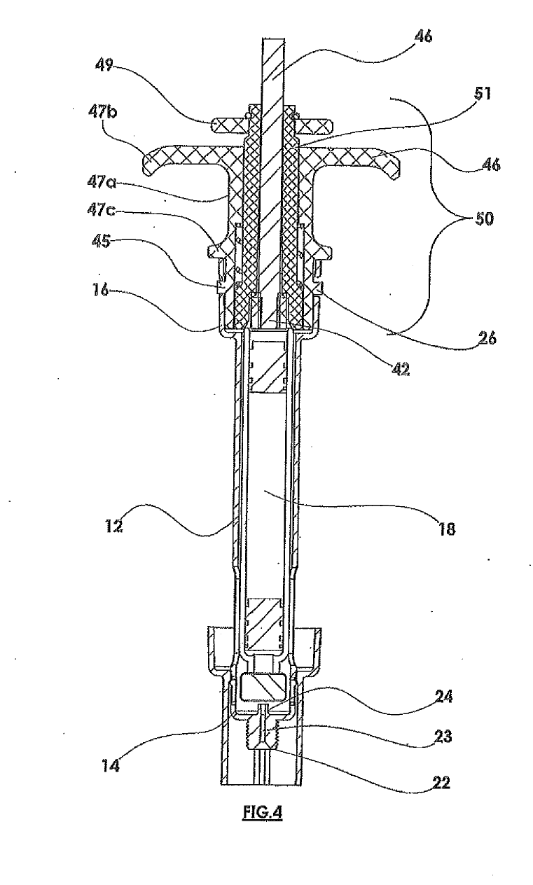

The tubular housing unit 20 comprises a barrel 12 adapted and sized to receive a medication-containing carpule 18 as shown in FIGS. 3 and 4. The front end 14 of the barrel 12 comprises a needle mounting means 22 onto which a needle unit can be securely and removably mounted. The needle mounting means 22 may be any means useful to stably attach a needle unit, including a threaded protrusion as shown. Alternatively, the barrel may be provided with a needle affixed thereto which is not removable. The needle mounting means 22 includes a passage 23 therethrough to receive the end of a needle, thereby permitting contact of the end of the needle with a penetrable membrane at the needle-contacting end of a carpule 18 within the barrel 12. The passage 23 extends or protrudes into the barrel 12 beyond the front end 14 of the barrel 12 in the form of projection 24 to provide a space (e.g. at least about 1 mm, and preferably at least 2-3 mm) between the carpule and the front end 14 of the barrel 12 that permits some forward movement of a carpule within the barrel 12 which is important following injection when the syringe is used to aspirate.

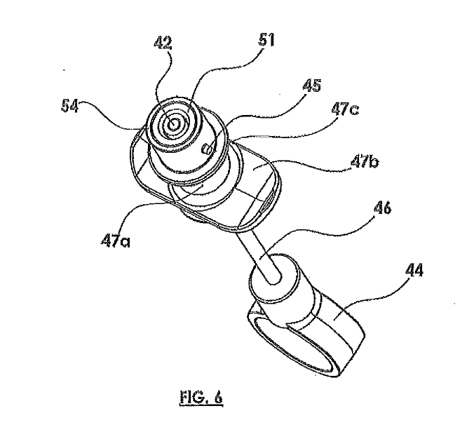

The rear end 16 of the barrel 12 has an opening 25 which permits loading of a carpule into the barrel 12. The rear end 16 and rear end opening 25 are also adapted to receive and releasably engage the plunger unit 40. In this regard, to provide attachment to the plunger unit 40 that is secure while also providing ease of assembly and disassembly by a user, the rear end 16 of the barrel 12 incorporates attachment means 26 that securely engage a barrel-receiving end 54 of the plunger unit 40 as best shown in FIG. 6. In one embodiment, a Luer lock attachment in which the rear end 16 of the barrel 12 incorporates one or more apertures is provided as attachment means 26 to securely engage corresponding projections 45 located at the barrel-receiving end 54 of the plunger unit 40. The apertures as attachment means 26 are shaped to receive projections 45 on the plunger unit 40 such that the housing unit 20 is securely attached to the plunger unit 40. In one embodiment, the apertures (attachment means) 26 comprise an angled shape, e.g. such as an “L” or similar shape. Such angled apertures receive a projection 45 and provide a secure attachment on application of a twisting motion such that the projection 45 is positioned at the end or tip of the base of the angled aperture and will not move longitudinally (to release the barrel from the plunger) without a reversed twisting action. To provide a more secure attachment, the rear end 16 of the barrel 12 may optionally incorporate a flange 28 to provide the rear end 16 with a circumference that is larger than that of the body of the barrel 12 which press fits onto the barrel-receiving end of the plunger 40. As one of skill in the art will appreciate, for stability, the rear end 16 of the barrel 12 (in which the apertures are formed) is of a sufficient thickness so as to maintain the integrity thereof, e.g. a thickness in the range of about 0.8-1.0 mm, such as about 0.92 mm +/−0.05 mm.

The barrel 12 may include cut outs or openings 29 formed in the sides thereof (as shown in FIG. 1) for ready visibility of a carpule situated within the barrel 12, and to assist with the insertion and removal of the carpule from the barrel 12. In this regard, such openings 29 permit a user to grip a carpule within the barrel 12 by squeezing the barrel at an opening with their fingertips to grip the carpule, and then removing the carpule by pulling the carpule towards the rear end 16 of the barrel.

The housing unit 20 also comprises a hollow protective sheath 30 as shown in FIG. 3, having a front end 32 and a rear end 33, which functions to cover or expose the needle, as desired. The sheath 30 is slideably mounted onto the barrel 12 and is slidable between a first extended position which extends past the front injection end of a needle to cover the end of the needle, and a second retracted position in which the needle is exposed for use. In the absence of a needle, the needle-mounting means 22 is exposed when the sheath 30 is in the retracted position to permit mounting of a needle thereon. The barrel 12 and/or the sheath 30 incorporates position-retaining means that releasably retain the sheath 30 in either its first extended position or its second retracted position. As one of skill in the art will appreciate, a variety of mechanisms may be employed as retaining means to releasably retain the sheath 30 in a desired position, including a twist and lock mechanism, a groove and ridge mechanism, threading, press fitting, or the like. For example, to retain the sheath 30 in the extended position (in which the sheath 30 covers the end of a needle attached to the barrel 12), the interior of the rear end 33 of the sheath 30 may incorporate threading to engage with corresponding threading formed on the exterior of the front end 14 of the barrel 12, or the interior of the rear end 33 of the sheath 30 and/or the exterior of the front end 14 of the barrel 12 may incorporate one or more ridge(s) and/or groove(s) to maintain the sheath 30 in the extended position. Likewise, the retracted position of the sheath 30 may be retained by threading or ridges/grooves formed in the interior of the rear end 33 of the sheath 30 to engage with corresponding threading or ridges/grooves on the exterior of the rear end 16 of the barrel 12.

In one embodiment, the exterior of the front end 14 of the barrel 12 incorporates a groove with a ridge on either side (partially or wholly about its circumference) which functions to engage the interior of the rear end 33 of sheath 30 (which may optionally also include a ridge to fit within the groove) when a pulling force of the sheath 30 relative to the barrel is applied in opposing directions (e.g. away from one another), and retains the sheath in an extended position. The extended position of the sheath 30 may be released by applying a pushing force of the sheath 30 relative to the barrel 12 in opposing directions (e.g. towards one another). To maintain the sheath in a retracted position, the rear end 33 of the sheath 30 incorporates a flange 38 that press fits onto the flange 28 of barrel 12 (i.e. the circumference of flange 38 with respect to flange 28 is only somewhat larger such that the retracted position of sheath 30 is retained by a tight or press fit.). The retracted position of the sheath 30 is released by applying a pulling force of the sheath 30 relative to the barrel 12 in opposing directions). The width of flange 38 of sheath 30 will be smaller than the width of flange 28 of barrel 12, e.g. about ½ the width such that it does not interfere with attachment means 26 on flange 28.

The sheath 30 may include cut-outs or openings 39 formed in the sides thereof which may generally line up with the cut-outs or openings 29 formed in the sides of the barrel 12 when the sheath 30 is in its retracted position. The cut-outs permit viewing of the carpule within the barrel 12 during use of the syringe, and may also provide access to the carpule for removal.

To prevent rotation of the barrel 12 within the sheath 30, the barrel and/or sheath may include means to maintain the position of the sheath 30 relative to the barrel 12. In one embodiment, the barrel 12 is provided with one or more ridges formed along the length of the exterior of barrel 12 which engage with corresponding grooves formed along the length of the interior of sheath 30.

The housing unit may optionally include a cap that fits onto the front end 32 of the sheath 30 to provide further protection from the injection end of a needle secured to the housing unit. The cap may be held in place in the usual manner, including, but not limited to, a twist lock mechanism, engagement with a dimple or depression, threading, or by a press fit.

The housing unit 20, including the barrel 12, sheath 30 and cap (if present) may be made of any suitable material, preferably a disposable material such as a polymeric material, e.g. plastic and the like. In one embodiment, the material used to make the barrel 12 and sheath 30 is transparent to permit viewing of the carpule contents, and particularly to determine the presence of any blood on aspiration (i.e. indicative that a blood vessel has been hit on injection).

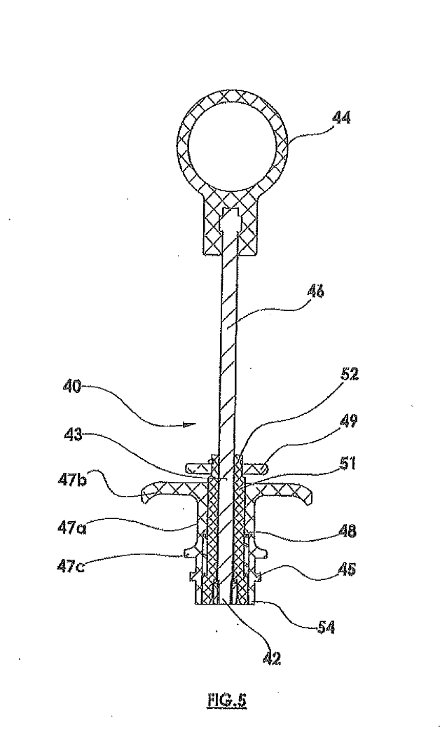

The syringe further comprises a plunger unit 40, an embodiment of which is shown in FIGS. 5 and 6. The plunger unit 40 comprises: (i) a plunger; (ii) a pusher housing; and (iii) an aspirator.

The plunger includes a plunger head 42, a thumb rest 44 and a shaft 46 connecting the plunger head 42 to the thumb rest 44 (FIG. 5). The thumb rest 44 may be any suitable thumb rest, including a thumb ring. In a preferred embodiment, the thumb rest 44 is ergonomically designed, preferably in the general shape of a thumb tip, e.g. an open-ended thumb pocket or pouch, which functions to engage and hold the thumb of a user in position. The user can comfortably insert his or her thumb tip into the thumb rest 44 and readily depress or retract the plunger head 42. The pocket advantageously holds the thumb in position as opposed to a thumb ring which requires the user to hold the thumb in position which results in fatigue in the thumb.

The pusher housing 50 comprises a plunger-receiving end 52 and a barrel-receiving end 54 (FIG. 4-6). The pusher housing 50 receives the plunger at the plunger-receiving end in a channel 43 formed within the pusher housing 50 that extends from the plunger-receiving end 52 to the barrel-receiving end 54. A finger rest 48 for the index finger and middle finger is formed on the exterior of the pusher housing 50 and includes a middle section 47a on which the fingers rest and upper and lower ridges 47b/c, respectively, at either end of the middle section 47a to maintain the fingers in position. The finger rest 48 provides support to the fingers when the syringe is being used and a downward pressure is applied by the thumb in thumb rest 44. In some embodiments, the upper ridge 47b may be more flange-like to provide more finger support, for example, upper ridge 47b may have a larger diameter than lower ridge 47c.

The pusher housing 50 includes one or more protrusions 45 at the barrel-receiving end 54 that are adapted to engage the attachment means 26 of the flange 28 of the barrel 12. Preferably, the pusher housing includes one or more protrusions 45 to engage corresponding attachment means 26 formed in opposite sides of the flange 28 of the barrel 12. Preferably, there are at least two protrusions 45 positioned at opposite sides of the barrel-receiving end 54 of the pusher housing to engage with corresponding attachment means 26.

The plunger unit 40 also includes spring-loaded aspirator which functions to permit the syringe to aspirate, i.e. result in suction by a pull or backward movement of the plunger away from a site of injection. The aspirator comprises an actuator ring 49 mounted on the shaft 46 of the plunger unit adjacent to the plunger-receiving end 52 of the pusher housing 50. The actuator ring 49 is mounted on the shaft 46 such that the shaft 46 is moveable through the actuator ring 49. A spring-loaded hollow piston 51 abuts the actuator ring 49 and is moveably seated within channel 43 of the pusher housing 50. The plunger shaft 46 is similarly seated within a channel formed in piston 51. The plunger head 42 at the barrel-receiving end of the pusher housing 50 has a diameter which is larger than that of the channel formed in piston 51, thereby retaining the pusher housing 50 on the plunger. A backward force (towards the thumb rest 44) applied by the index and middle fingers to the actuator ring 49 results in a backward movement of the piston which causes the plunger also to move backward, resulting in aspiration when the syringe is in use as further described below.

The plunger head 42 is adapted to engage with the seal of a carpule situated within the barrel 12 of the housing unit 20 such that when a force is applied to the shaft 46 of the plunging unit 40, the plunger head 42 will move the carpule seal within the carpule. Accordingly, the plunger head 42 is designed to engage the end of the carpule, and may include a protrusion to be received by the carpule seal, an aperture to be engaged by the carpule seal, or a blunt end.

The plunger unit 40 may be made of any suitable material, including a disposable or non-disposable medical-grade material. Preferably, the plunger unit 40 is made of an autoclavable material to permit re-use. Examples of suitable autoclavable materials include, but are not limited to, metals such as stainless steel, metal alloys and other moldable materials.

The syringe 10 is for use with a needle unit which includes a needle, such as a double-end hypodermic needle (e.g. ½ inch, 1 inch or 1¾ inch needle) of varying gauges (e.g. 27 or 30 gauge). The needle includes a front injection end and a rear end, as well as an attachment means to connect the needle to the housing unit 20 via the mounting means 22 on the barrel 12 of the syringe 10. Alternatively, the syringe may be provided with a needle already affixed thereto. The rear end of the needle extends through the channel 23 formed in the attachment means 22 of the barrel 12 and penetrates the membrane at the front end of a carpule within the barrel. Thus, once the rear end of the needle penetrates the carpule membrane, the needle is in fluid communication with the liquid medicine inside the carpule. The channel 23 is preferably of a circumference that tightly maintains the selected needle in position and does not allow side to side movement of the needle within the syringe.

In use, a carpule 18 is loaded into the barrel 12 of the housing unit 20 through the rear end opening 25 of barrel 12. The sheath 30 of the housing unit 20 may either be in the extended or retracted position for carpule loading. However, if the housing unit 20 is provided with a needle already affixed thereto, the sheath will be in the extended position to cover the end of the needle. The barrel 12 is then attached to the plunger unit 40. The flange 28 of the barrel 12 with attachment means (e.g. angled apertures) 26 is slid onto the barrel receiving end 54 of the plunger unit 40 such that apertures 26 engage protrusions 45 followed by a twisting motion to affix the housing unit 20 to the plunger unit 40. Attachment is confirmed by applying a pulling force of the housing unit 20 relative to the plunger unit 40 in opposing directions.

Once the housing unit 20 and plunger unit 40 are attached, and a needle is to be mounted, the sheath 30 of the housing unit 20 is put into its retracted position, and a needle unit is attached to the housing unit 20 by inserting the end of the needle into the needle mount means 22 through channel 23 and into the penetrable membrane of the carpule 18. If the syringe is not to be used immediately, the sheath 30 is moved into its extended position to cover the front end of the needle. A cap may be put on the front end of the sheath 30 for greater protection from unintentional needle sticks. Alternatively, the needle may have a covering cap that the sheath 30 can slide over without interference.

To prepare the syringe for delivering an injection to a patient, the sheath cap, if present, is removed. To expose the needle, the sheath 30 is moved from the extended position to the retracted position by sliding the sheath along the barrel 12 of the housing unit 20 to engage the rear end 33 of sheath 30 with the rear end 16 of the barrel 12 via retaining means. If the needle is covered, then this covering is removed.

A user will then position the syringe appropriately for an injection, placing index and middle finger on the finger rest 48 and thumb in the thumb rest 44, and applying pressure with the thumb, depresses the shaft 46 of the plunger unit 40. The plunger head 42 pushes against the carpule seal/stopper, which results in fluid from the carpule being expelled from the needle. Once an initial testing of the syringe is complete, the syringe may then be used for injection into a patient. Following the initial stick, the user may engage (pull up on) the spring-loaded actuator 49 to aspirate, viewing the carpule for aspiration of blood, and in the absence of blood continue with the injection by depressing the shaft 46 of the plunger unit 40 with the thumb.

On completing the injection, the syringe is then prepared for safe disposal. The front end of the needle may be covered. The sheath 30 is slid back into the extended position to cover the end of the needle by gripping the sheath 30 at its rear end 33, which is advantageously safely distanced from the injection end of the needle, thereby minimizing the risk of an accidental needle stick. An appropriate pulling/pushing force is applied to disengage retaining means and the sheath is moved from the retracted position to the extended position. Retaining means at the front end of the barrel 12 are engaged to retain the sheath 30 in the extended position. The cap 34, if used, may then be placed onto the front end of the sheath 30. The plunger unit 40, if re-useable, may be detached from the rear end 16 of the barrel 12 by disengaging the attachment means from the barrel receiving means for re-use following proper sterilization protocol. The housing, carpule and needle unit are then safely disposed of without incident.

The scope of the claims should not be limited by the preferred embodiments set forth in the examples, but should be given the broadest interpretation consistent with the description as a whole.

1. A plunging unit for a syringe comprising:
(i) a plunger comprising a plunger head connected to a thumb rest by a shaft;
(ii) a pusher housing comprising a finger rest and an internal channel connecting a plunger-receiving end of the housing to a barrel-receiving end of the housing, wherein the plunger-receiving end is adapted to receive the plunger head into the channel and the barrel-receiving end comprises one or more protrusions adapted to engage a barrel of a carpule housing unit; and
(iii) optionally, an aspirator adapted to cause a backward movement of the plunger away from a site of injection, wherein the aspirator comprises an actuator ring on the shaft of the plunger adjacent to the plunger-receiving end of the housing, and when the actuator ring is moved in a backward direction, a spring-loaded piston that sits within the pusher housing and houses the shaft of the plunger is pulled backward which causes the plunger to move backward.

2. The plunging unit of claim 1, wherein the pusher housing comprises at least two protrusions adapted to engage the barrel of a carpule housing unit.

3. The plunger unit of claim 1, wherein the plunger head is adapted to engage the end of a medication-containing carpule.

4. The plunger unit of claim 1, made of a non-disposable autoclavable material.

5. A kit comprising a plunging unit as defined in claim 1, and one or more housing units, wherein the housing units comprise:
i) a barrel adapted to receive a carpule, said barrel having a front end comprising a needle mounting means, and a rear end comprising an opening to receive a carpule and attachment means to releasably engage with the protrusions on pusher housing of the plunging unit; and
ii) a sheath having a front end and a rear end, said sheath being slidably mounted onto the barrel, said sheath being moveable between a first extended position to cover a needle mounted on the barrel and a second retracted position which exposes a needle mounted on the barrel.

6. The kit as defined in claim 5, wherein the attachment means in the rear end of the barrel is one or more angled apertures.

7. The kit as defined in claim 6, wherein the angled apertures are L-shaped.

8. The kit as defined in claim 6, wherein the pusher housing of the plunging unit comprises at least two protrusions adapted to engage the barrel of the housing unit, and the rear end of the barrel comprises corresponding angled apertures for each protrusion.

9. The kit as defined in claim 5, wherein the needle-mounting means on the barrel includes a channel therethrough for receiving a needle end into the barrel.

10. The kit as defined in claim 9, wherein the channel forms a protrusion within the barrel to provide a space between the carpule and the front end of the barrel.

11. The kit as defined in claim 5, wherein the rear end of the barrel comprises a flange.

12. The kit as defined in claim 5, wherein at least one of the barrel and sheath incorporates position retaining means that releasably retain the sheath in its first extended position or second retracted position.

13. The kit as defined in claim 12, wherein the position retaining means comprises a mechanism selected from the group consisting of a twist and lock mechanism, a groove and ridge mechanism, threading and a press fit.

14. The kit as defined in claim 13, wherein the front end of the barrel incorporates a groove with a ridge on either side at least partially about its circumference to retain the sheath in its extended position.

15. The kit as defined in claim 13, wherein the rear end of the sheath incorporates a flange that fits onto a flange at the rear end of the barrel to retain the sheath in a retracted position.

16. The kit as defined in claim 5, wherein the barrel and/or sheath include means to prevent rotation of the sheath relative to the barrel.

17. The kit as defined in claim 16, wherein the barrel incorporates one or more ridges or grooves along its length to engage with one or more corresponding grooves or ridges formed in the sheath along its length.

18. The kit as defined in claim 5, further comprising one or more needles.
